(19) United States
(12) Patent Application Publication
(10) Pub. No.: US 2025/0107991 A1
(43) Pub. Date: Apr. 3, 2025

(54) COMPOSITION COMPRISING GLUCOLIPIDS

(71) Applicant: Evonik Operations GmbH, Essen (DE)

(72) Inventors: Stefan Julian Liebig, Duesseldorf (DE); Jochen Kleinen, Heinsberg (DE); Hans Henning Wenk, Muelheim an der Ruhr (DE); Karin Fürch, Essen (DE); Marius Merschmann, Essen (DE); Jakob Müller, Haltern am See (DE); Hacer Yalcinkaya, Essen (DE); Stefanie Volkmer, Essen (DE)

(73) Assignee: Evonik Operations GmbH, Essen (DE)

(21) Appl. No.: 18/715,171

(22) PCT Filed: Nov. 25, 2022
(86) PCT No.: PCT/EP2022/083254

§ 371 Date: May 31, 2024

(30) Foreign Application Priority Data
Dec. 2, 2021 (EP) 21211919.2

(51) Int. Cl. A61K 8/60; A01N 25/02; A01P 17/00; A61Q 19/00

(52) U.S. Cl. CPC A61K 8/602; A01N 25/02; A01P 17/00; A61Q 19/00; A61K 2800/10

(57) ABSTRACT

A composition includes 20% to 70% by weight of at least one glucolipid of formula (I) or salts thereof and 30% to 80% by weight of water. A pH of the composition at 25° C. is from 3.5 to 8.0. A method prepares a formulation containing glucolipids by mixing a composition containing glucolipids with at least one further formulation component and adjusting the glucolipid content by adding water.

### FIELD OF THE INVENTION

The invention relates to compositions comprising glucolipids and a method for preparing formulations containing glucolipids.

### PRIOR ART

Glycolipids can be obtained as metabolic products of certain microorganisms. Both wildtype strains and genetically modified strains are used as microbial production hosts.

Glycolipids are surface-active substances and due to their biodegradability are therefore interesting for a wide range of technical applications, especially in the personal and household care field, but also in agro- or fracking applications, as environmentally friendly alternative.

A product form that is liquid at room temperature has proven to be the best choice for most of these applications. The low viscosity ensures processing via piping systems and pumps. On the other hand, a high concentration of active substances is required to keep the water content low. This enables the production of environmentally friendly formulations and contributes to ecological aspects, such as reduced fuel consumption per kg of active ingredient during transport.

In addition, a high active content facilitates preservation and thus the amount of preservative to be used per kg of active ingredient is decreased. Also, a high active content allows greater flexibility in formulation.

Matsuyama et al. in Serrawettins and Other Surfactants Produced by Serratia, Biosurfactants. Microbiology Monographs, vol 20, 2011 disclose the structure of the glycolipids rubiwettin RG1 and beta-D-glucopyranosyl 3-(3′-hydroxytetradecanoyloxy)decanoate produced by Serratia marcescens.

DE19648439 discloses the use of mixtures of glycolipids and surfactants for the production of manual dishwashing detergents.

WO2019154970 discloses mixture compositions comprising certain glucolipids, their use for producing formulations and formulations comprising these mixture compositions.

Rhamnolipids are readily commercially available as glycolipids that have been produced by fermentative processes.

EP3023431 discloses concentrated rhamnolipid compositions and their preparation. However, the products described here show a significantly increased viscosity at high concentrations. Furthermore, the rhamnolipid containing compositions of EP3023431 cannot be adjusted to pH values below pH 5.5 without obtaining paste-like masses, just as disclosed in example 2 of EP3023431.

Cosmetic formulations, however, are frequently preserved by acids, resulting in a lower pH in the formulation. The same is true for a lot of cleaning formulations, where acidic components are incorporated as cleaning active, for example to dissolve calciferous sullage.

Consequently, there is a need for highly concentrated glycolipid compositions which bear low viscosities, even at low pH.

Preferably these glycolipids have a simple structure and/or a lower molecular weight to facilitate production and handling.

### DESCRIPTION OF THE INVENTION

It was found that, surprisingly, that certain glucolipids can be concentrated to high levels and bear very low viscosities at low pH values and are also stable at pH values as low as 4.0.

The present invention therefore provides compositions comprising glucolipids as described in claim 1.

The invention further provides and a method for preparing formulations containing glucolipids as described in claim 11.

One advantage of the present invention is the homogeneity of the products even at low pH values. Another advantage of the present invention is that the compositions are easier to dilute.

Another advantage of the present invention is that the compositions have an increased microbiological stability.

Another advantage of the present invention is that the compositions are easily miscible with other surfactants.

A further advantage of the present invention is that it is easier to incorporate into cosmetic formulations.

Another advantage of the present invention is that the compositions allow the formulation of concentrated surfactant formulations, even at very low pH.

Another advantage of the present invention is that the compositions have a reduced foam tendency due to their high concentration and the low achievable pH value, thus simplifying transport and delivery.

Another advantage of the present invention is that the salts of the instant invention have superior foam stabilizing properties as aqueous solutions.

Another advantage of the present invention is that the compositions allow for easy incorporation of hydrophobic components such as oils.

Another advantage of the present invention is that the compositions have a high storage stability. A further advantage of the present invention is that the compositions cause less contamination during their production and transport in pipelines and, moreover, allow easier cleaning.

Another advantage of the present invention is that the compositions require less energy for transport.

Another advantage of the present invention is that the compositions have a very low freezing point, which means that the compositions remain processable even at low temperatures.

In connection with the present invention, the term “glucolipid” is understood as meaning compounds of the general formula (I) or salts thereof,

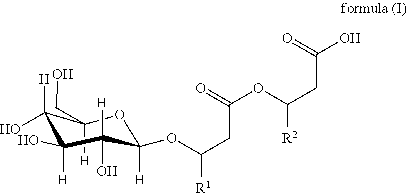

where
m=1 or 0,
R¹ and R²=independently of one another identical or different organic radical having 2 to 24 carbon atoms, in particular optionally branched, optionally substituted, in particular hydroxy-substituted, optionally unsaturated, in particular optionally mono-, di- or triunsaturated, alkyl radical, preferably one selected from the group consisting of pentenyl, heptenyl, nonenyl, undecenyl and tridecenyl and (CH₂)ₒ—CH₃ where o=1 to 23, preferably 4 to 12.

Distinct glucolipids are abbreviated according to the following nomenclature:
“GL-CXCY” is understood as meaning glucolipids of the general formula (I) in which one of the radicals R¹ and R²═(CH₂)ₒ—CH₃ where o=X-4 and the remaining radical R¹ or R²═(CH₂)ₒ—CH₃ where o=Y-4.

The nomenclature used thus does not differentiate between “CXCY” and “CYCX”.

If one of the aforementioned indices X and/or Y is provided with “:Z”, then this means that the respective radical R¹ and/or R²=an unbranched, unsubstituted hydrocarbon radical with X-3 or Y-3 carbon atoms having Z double bonds.

The present invention provides a composition comprising
20% by weight to 70% by weight, preferably 35% by weight to 60% by weight, particularity preferably 40% by weight to 50% by weight, of at least one glucolipids of the general formula (I) or salts thereof

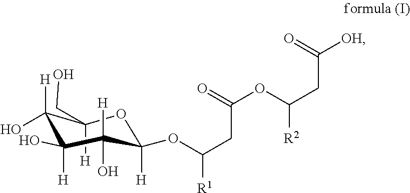

where
R¹ and R²=independently of one another identical or different organic radical having 2 to 24 carbon atoms,
and
30% by weight to 80% by weight, preferably 40% by weight to 65% by weight, particularity preferably 50% by weight to 60% by weight, of water,
where the percentages by weight refer to the total composition, characterized in that the pH of the composition at 25° C. is from 3.5 to 8.0, preferably from 3.8 to 6.9, more preferably from 4.1 to 6.1 and particularity preferably from 4.5 to 5.4.

The “pH” in connection with the present invention is defined as the value which is measured for the relevant composition at 25° C. after stirring for 5 minutes using a calibrated pH electrode in accordance with ISO 4319 (1977).

The term“total dry mass” in the context of the present invention is understood to mean the portion of the composition according to the invention which remains—naturally in addition to water—after the composition according to the invention has been freed of the components which are liquid at 25° C. and 1 bar.

To determine the content of glucolipids in the context of the present invention, only the mass of the glucolipid anion is considered, i.e. “general formula (I) less one hydrogen”.

To determine the content of glucolipids in the context of the present invention, all glucolipids are converted by acidification into the protonated form (cf. general formula (I)) and quantified by HPLC. Unless otherwise stated, all percentages (%) given are percentages by weight.

A preferred composition according to the invention is characterized in, that the composition comprises at least 51% by weight to preferably 98% by weight, preferably 60% by weight to 95% by weight, more preferably 70% by weight to 90% by weight, particularly preferably 75% by weight to 85% by weight, glucolipids GL-C10C10 of the general formula (I) with R¹ and R²═(CH₂)₆—CH₃, where the percentages by weight refer to the sum of all of the glucolipids of the general formula (I) present.

A preferred composition according to the invention is characterized in, that it comprises at least 60% by weight, preferably at least 80% by weight, particularly preferably at least 90% by weight, of glucolipids of the general formula (I), where the percentages by weight refer to the total dry mass of the overall composition.

The glucolipids present in the compositions according to the invention are present at least partially as salts on account of the given pH.

In preferred compositions according to the instant invention the cations of the glucolipid salts present are selected from the group comprising, preferably consisting of, Li⁺, Na⁺, K⁺, Mg²⁺, Ca²⁺, Al³⁺, NH₄⁺, Zn²⁺, primary ammonium ions, secondary ammonium ions, tertiary ammonium ions and quaternary ammonium ions and amino acids, preferably proteinogenic amino acids.

Exemplary representatives of suitable ammonium ions are tetramethylammonium, tetraethylammonium, tetrapropylammonium, tetrabutylammonium and [(2-hydroxyethyl)trimethylammonium] (choline) and also the cations of 2-aminoethanol (ethanolamine, MEA), diethanolamine (DEA), 2,2′,2″-nitrilotriethanol (triethanolamine, TEA), 1-aminopropan-2-ol (monoisopropanolamine), ethylenediamine, diethylenetriamine, triethylenetetramine, tetraethylenepentamine, 1,4-diethylenediamine (piperazine), aminoethylpiperazine and aminoethylethanolamine.

Particularly preferred cations are selected from the group comprising, preferably consisting of, Na⁺, Li⁺, K⁺, Ca²⁺, Mg²⁺, NH₄⁺ and the tetraethylammonium cation, with Li⁺, K⁺, Ca²⁺, Mg²⁺, NH₄⁺ and the tetraethylammonium cation being most preferred.

Mixtures of the abovementioned cations may also be present as cations of the glucolipid salts present according to the invention.

A preferred composition according to the invention is characterized in, that it comprises 50% by weight to 99% by weight, preferably 70% by weight to 95% by weight, particularly preferably 85% by weight to 90% by weight, of glucolipid anions, where % by weight refers to all anions except OH⁻ present in the composition.

It may be advantageous and is therefore preferred if the composition according to the invention comprises
1% by weight to 30% by weight, preferably 5% by weight to 25% by weight, particularly preferably 10% by weight to 20% by weight, of GL-C8C10,
where the percentages by weight refer to the sum of all of the glucolipids of the general formula (I) present.

A preferred composition according to the invention is characterized in, that the composition comprises
0.5% by weight to 20% by weight, preferably 3% by weight to 17% by weight, particularly preferably 5% by weight to 15% by weight, of GL-C10C12:1,
where the percentages by weight refer to the sum of all of the glucolipids of the general formula (I) present.

A further preferred composition according to the invention is characterized in that the composition comprises
0.5% by weight to 20% by weight, preferably 2% by weight to 15% by weight, particularity preferably 3% by weight to 12% by weight, of GL-C10C12
where the percentages by weight refer to the sum of all of the glucolipids of the general formula (I) present.

A particularly preferred composition according to the invention is characterized in that the composition comprises
1% by weight to 30% by weight, preferably 5% by weight to 25% by weight, particularly preferably 10% by weight to 20% by weight, of GL-C8C10,
0.5% by weight to 20% by weight, preferably 3% by weight to 17% by weight, particularly preferably 5% by weight to 15% by weight, of GL-C10C12:1,
0.5% by weight to 20% by weight, preferably 2% by weight to 15% by weight, particularly preferably 3% by weight to 12% by weight, of GL-C10C12
where the percentages by weight refer to the sum of all of the glucolipids of the general formula (I) present.

A very particularly preferred composition according to the invention is characterized in that the composition comprises
10% by weight to 20% by weight, of GL-C8C10,
5% by weight to 15% by weight, of GL-C10C12:1,
3% by weight to 12% by weight, of GL-C10C12
where the percentages by weight refer to the sum of all of the glucolipids of the general formula (I) present.

Over and above this, it is preferred if the composition according to the invention comprises glucolipids of the formula GL-CX in only small amounts. In particular, the composition according to the invention comprises preferably
0% by weight to 5% by weight, preferably 0.01% by weight to 4% by weight, particularity preferably 0.1% by weight to 3% by weight, of GL-C10,
where the percentages by weight refer to the sum of all of the glucolipids of the general formula (I) present.

The compositions according to the invention—due to their low viscosity, pourability and pumpability—can advantageously be incorporated into formulations, in particular into cosmetic and household care formulations.

Consequently, a further subject matter of the present invention is a method for preparing a formulation, in particular a cosmetic or household care formulation, containing glucolipids comprising the steps of
a) providing a composition comprising glucolipids according to the instant invention,
b) providing at least one further formulation component,
c) mixing the composition comprising glucolipids and the at least one further formulation component and adjusting the glucolipid content of the total composition to 0.5% by weight to 20% by weight, preferably 2.0% by weight to 15% by weight, particularity preferably 3.0% by weight to 12% by weight, of glucolipids by the addition of water, wherein the weight percentages refer to the total formulation.

Preferred compositions comprising glucolipids provided in method step a) are those compositions according to the instant invention, which are described as preferred compositions according to the instant invention above.

The at least one further formulation component provided in methods step b) is preferably selected from the group of
emollients,
emulsifiers,
thickeners/viscosity regulators/stabilizers,
UV photoprotective filters,
antioxidants,
hydrotropes (or polyols),
solids and fillers,
film formers,
pearlescent additives,
deodorant and antiperspirant active ingredients,
insect repellents,
self-tanning agents,
preservatives,
conditioners,
perfumes,
dyes,
odour absorbers,
cosmetic active ingredients,
care additives,
superfatting agents,
surfactants,
solvents.

Substances which can be used as exemplary representatives of the individual groups are known to the person skilled in the art and can be found for example in the German application DE 102008001788.4. This patent application is hereby incorporated by reference and thus forms part of the disclosure.

As regards further optional components and the amounts of these components used, reference is made expressly to the relevant handbooks known to the person skilled in the art, for example K. Schrader, “Grundlagen und Rezepturen der Kosmetika [Fundamentals and Formulations of Cosmetics]”, 2nd edition, page 329 to 341, Hüthig Buch Verlag Heidelberg.

The amounts of the respective additives are dependent on the intended use.

Typical guide formulations for the respective applications are known prior art and are contained for example in the brochures of the manufacturers of the respective base materials and active ingredients. These existing formulations can generally be adopted unchanged. If required, however, the desired modifications can be undertaken without complication by means of simple experiments for the purposes of adaptation and optimization.

The present invention furthermore provides, salts of at least one glucolipid, characterized in that it comprises at least one cation selected from the group comprising, preferably consisting of, Na⁺, Li⁺, K⁺, Mg²⁺, Ca²⁺, Al³⁺, NH₄⁺, primary ammonium ions, secondary ammonium ions, tertiary ammonium ions and quaternary ammonium ions.

The salts according to the invention preferably comprise at least 50% by weight, preferably at least 70% by weight, particularly preferably at least 95% by weight of the at least one cation, where the percentages by weight arise from the weight of the total salt “glucolipid anion plus cation” and refer to the total salt.

Exemplary representatives of suitable ammonium ions are tetramethylammonium, tetraethylammonium, tetrapropylammonium, tetrabutylammonium and [(2-hydroxyethyl)trimethylammonium] (choline) and also the cations of 2-aminoethanol (ethanolamine, MEA), diethanolamine (DEA), 2,2′,2″-nitrilotriethanol (triethanolamine, TEA), 1-aminopropan-2-ol (monoisopropanolamine), ethylenediamine, diethylenetriamine, triethylenetetramine, tetraethylenepentamine, 1,4-diethylenediamine (piperazine), aminoethylpiperazine and aminoethylethanolamine.

Particularity preferred cations are selected from the group comprising, preferably consisting of, Na⁺, Li⁺, K, Ca²⁺, Mg²⁺, NH₄⁺ and the tetraethylammonium cation, with Li⁺, K, Ca²⁺, Mg²⁺, NH₄⁺ and the tetraethylammonium cation being most preferred.

With respect to their glucolipid composition with regard to mono-, di- and fatty acid content, preferred glucolipid salts according to the invention have the above cited preferred glucolipids present in the compositions according to the invention.

The examples adduced hereinafter describe the present invention by way of example, without any intention that the invention, the scope of application of which is apparent from the entirety of the description and the claims, be restricted to the embodiments specified in the examples.

### EXAMPLES

As already discussed above, the object of the invention was to provide highly concentrated glycolipid compositions which bear low viscosities, even at low pH.

EP3023431 has also the objective to provide highly concentrated glycolipid compositions and achieves this by compositions comprising certain rhamnolipids.

In the following examples it is clearly proven, that the glucolipids of the instant invention outperform the compositions of EP3023431 by far.

### Example 1: Production of a Highly Concentrated Glucolipid Solution

Glucolipids were produced according to example 2 of WO2019154970 via fermentation.

Cells were separated by centrifugation at 10.000 g for 20 minutes. The fermentation broth was separated as the supernatant and adjusted to pH 3.1 by addition of concentrated H₂SO₄.

After a second centrifugation at 5.000 g for 20 minutes the aqueous upper phase was separated off and the remaining lower phase was a concentrate, which had a content of more than 50 wt.-% of glucolipids.

### Example 2: Partial Neutralisation with KOH

The glucolipid concentrate of example 1 was adjusted to different pH values by the addition of 50 wt.-% KOH (aq) under constant stirring as depicted in table 1.

Further, different glucolipid concentrations as depicted in table 1 were produced by the addition of water.

The viscosity was measured using a rheometer (MCR 302, Anton Paar Germany) in a parallel plate measuring system. The upper plate had a diameter of 40 mm, the gap distance was 0.5 mm, measuring temperature was 25° C. The measurement was conducted at a shear rate of 100 s⁻¹.

The results are shown in table 1.

It was surprisingly found, that by partial neutralization of the glucolipids highly concentrated compositions with very low viscosities can be obtained.

TABLE 1
Viscosities (Pas, shear rate 100 s⁻¹) of glucolipid
compositions at different concentrations and pH values.

| | 20% | 30% | 40% | 50% |
|---|---|---|---|---|
| pH 4.5 | 0.312 | 0.389 | 0.618 | 1.23 |
| pH 6 | 0.0589 | 0.154 | 0.320 | 0.425 |
| pH 7 | 0.00641 | 0.042 | 0.234 | 0.459 |

These viscosities found are way lower than those of the corresponding solutions of rhamnolipids, as disclosed in EP3023431 (see table below), which overall have a similar fatty acid residue distribution:

| | 20% | 30% | 40% | 50% |
|---|---|---|---|---|
| pH 3.5 | — | — | 6.04 | |
| pH 5.6 | 0.406 | 0.593* | 0.816* | 1.756* |
| pH 6.0 | 0.12 | 0.536* | 0.922* | 1.698* |
| pH 7.0 | 0.014 | 0.212 | 1.010 | 1.793 |

### Example 3: Surface Tension

### Description of the Method

Surface tensions were measured with the Dataphysics OCA 25 instrument based on the pendant drop method. Measurements were performed with 0.5% concentration of surfactant in water at 22° C. Measurement duration is standardized as 300 seconds and each measurement was repeated twice.
Glucolipid of example 2 (concentration: 0.5%)
Surface Tension (pH 5.9): 25.6 mN/m
Surface Tension (pH 7): 30.9 mN/m
Di-rhamnolipid (concentration: 0.5%)
Surface Tension (pH 5.5): 28.0 mN/m
Surface Tension (pH 7): 32 mN/m

The data shows, that the glucolipids of the instant invention have slightly superior surface activity than the corresponding di-rhamnolipids.

### Example 4: Sodium Sat of Glucolipid (According to the Invention)

The highly concentrated, acidic glucolipid suspension obtained in example 1 was adjusted by addition of a 50% by weight NaOH solution until a pH of 6 was reached. Then water was added to obtain a glucolipid content of 30.0% by weight.

### Example 5: Ammonium Salt of Glucolipid (According to the Invention)

The highly concentrated, acidic glucolipid suspension obtained in example 1 was adjusted by addition of a 25% by weight NH₄OH solution until a pH of 6 was reached. Then water was added to obtain a glucolipid content of 30.0% by weight.

### Example 6: Lithium Sat of Glucolipid (According to the Invention)

The highly concentrated, acidic glucolipid suspension obtained in example 1 was adjusted by addition of a 25% by weight LiOH solution until a pH of 6 was reached. Then water was added to obtain a glucolipid content of 30.0% by weight.

### Example 7: Tetraethylammonium Salt of Glucolipid (According to the Invention)

The highly concentrated, acidic glucolipid suspension obtained in example 1 was adjusted by addition of a 35% by weight N(Et)₄OH solution until a pH of 6 was reached. Then water was added to obtain a rhamnolipid content of 30.0% by weight.

### Example 8: Potassium Salt of Glucolipid (According to the Invention)

The highly concentrated, acidic glucolipid suspension obtained in example 1 was adjusted by addition of a 50% by weight KOH solution until a pH of 6 was reached. Then water was added to obtain a glucolipid content of 30.0% by weight.

### Example 9: Calcium Salt of Glucolipid (According to the Invention)

The highly concentrated, acidic glucolipid suspension obtained in example 1 was adjusted by addition of a 25% by weight Ca(OH)₂ solution until a pH of 6 was reached. Then water was added to obtain a glucolipid content of 30.0% by weight.

### Example 10: Magnesium Salt of Glucolipid (According to the Invention)

The highly concentrated, acidic glucolipid suspension obtained in example 1 was adjusted by addition of a 25% by weight Mg(OH)₂ solution until a pH of 6 was reached. Then water was added to obtain a glucolipid content of 30.0% by weight.

### Example 11: Potassium Salt of Di-Rhamnolipid (not According to the Invention)

A highly concentrated, acidic rhamnolipid suspension was adjusted by addition of a 50% by weight KOH solution until a pH of 6 was reached. Then water was added to obtain a Rhamnolipid content of 30.0% by weight.

### Example 12: Foam Stability of Glucolipid Salts with Monovalent Cations

Formulations were prepared in a 150 mL beaker by mixing 1.67 g of each sample (examples 4, 5, 6 7, 8 and 11) with 98.33 g of water leading to surfactant concentrations of 0.5%. Then 20 mL of the diluted samples were transferred to a 100 mL measuring cylinder. Foam was generated by shaking the closed measuring cylinder forty times. The foam height was observed for one hour. The foam height is given in mL.

| | | Foam volume in mL | | | | | |
|---|---|---|---|---|---|---|---|
| | | 0 | 1 | 5 | 15 | 30 | 60 |
| | sample name | min | min | min | min | min | min |
| 4 | Na⁺ glucolipid | 100 | 99 | 100 | 98 | 100 | 100 |
| 5 | NH₄⁺ glucolipid | 100 | 100 | 100 | 100 | 100 | 100 |
| 6 | Li⁺ glucolipid | 120 | 110 | 110 | 100 | 110 | 100 |
| 7 | N(CH₂CH₃)₄⁺ glucolipid | 110 | 110 | 110 | 110 | 110 | 110 |
| 8 | K⁺ glucolipid | 110 | 110 | 110 | 110 | 110 | 110 |
| 11 | K⁺ di-rhamnolipid | 95 | 93 | 93 | 93 | 93 | 93 |

The data shows, that the glucolipid salts of the instant invention have superior foam stability than the corresponding di-rhamnolipids salts.

### Example 13: Foam Stability of Glucolipid Salts with Divalent Cations

Formulations were prepared in a 150 mL beaker by mixing 1.67 g of each sample (examples 9, 10 and 11) with 98.33 g of water leading to surfactant concentrations of 0.5%. Then 50 mL of the diluted samples were transferred to a 250 mL measuring cylinder. Foam was generated by shaking the closed measuring cylinder forty times. The foam height was observed for one hour. The foam height is given in mL.

| | | Foam volume in mL | | | | | |
|---|---|---|---|---|---|---|---|
| | | 0 | 1 | 5 | 15 | 30 | 60 |
| | sample name | min | min | min | min | min | min |
| 9 | Ca₂⁺ glucolipid | 300 | 300 | 300 | 270 | 260 | 250 |
| 10 | Mg₂⁺ glucolipid | 300 | 300 | 300 | 300 | 300 | 290 |
| 11 | K⁺ di-rhamnolipid | n.d. | 250 | 250 | 230 | 220 | 200 |

The data shows, that the glucolipid salts of the instant invention have superior foam stability than the corresponding di-rhamnolipids salts.

### Example 14: Viscosities of High Concentrated Glucolipid Salts

The obtained 30% concentrated glucolipid salts from example 4 (30% Na⁺ glucolipid salt in water, pH6), example 5 (30% NH₄⁺ glucolipid salt in water, pH6), example 6 (30% Li⁺ glucolipid salt in water, pH6), example 7 (30% N(CH₂CH₃)₄⁺ salt in water, pH6) and example 10 (30% Mg²⁺ glucolipid in water, pH6) were analyzed on viscosity.

The viscosity was measured using a rheometer (MCR 302, Anton Paar Germany) in a parallel plate measuring system. The upper plate had a diameter of 40 mm, the gap distance was 0.5 mm, measuring temperature was 25° C. The measurement was conducted at a shear rate of 100 s⁻¹.

| | Viscosity in |
|---|---|
| | Pa*s at 25° C., |
| Sample | shear rate 100 s⁻¹ |
| 30% K⁺ di-rhamnolipid salt in water, pH 6 | 0.536 |
| (from EP3023431, compare above in example 2) | |
| 30% K⁺ glucolipid salt in water, pH 6 (example 2) | 0.154 |
| 30% Na⁺ glucolipid salt in water, pH 6 (example 4) | 0.314 |
| 30% NH₄⁺ glucolipid | 0.271 |
| salt in water, pH 6 (example 5) | |
| 30% Li⁺ glucolipid salt in water, pH 6 (example 6) | 0.312 |
| 30% N(CH₂CH₃)₄⁺ | 0.214 |
| salt in water, pH 6 (example 7) | |
| 30% Mg²⁺ glucolipid in water, pH 6 (example 10) | 0.393 |

The here shown mono and divalent glucolipid salts have all a lower viscosity compared to the potassium di-rhamnolipid sat at the same concentration and pH.

1. A composition, comprising:
20% by weight to 70% by weight of at least one glucolipid of formula (I) or salts thereof 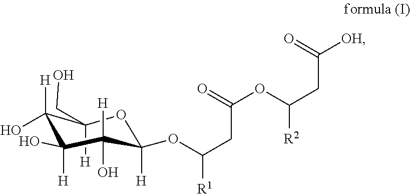
wherein R¹ and R² are independently of one another identical or different organic radicals having 2 to 24 carbon atoms, and
30% by weight to 80% by weight of water,
wherein the percentages by weight refer to a total weight of the composition, wherein a pH of the composition at 25° C. is from 3.5 to 8.0.

2. The composition according to claim 1, comprising:
at least 51% by weight glucolipids GL-C10C10 of formula (I), wherein R¹ and R² are (CH₂)₆—CH₃, wherein the percentages by weight refer to a sum of all of the glucolipids of formula (I) present.

3. The composition according to claim 1, comprising:
at least 60% by weight of glucolipids of formula (I), wherein the percentages by weight refer to a total dry mass of the overall composition.

4. The composition according to claim 1, wherein cations of the glucolipid salts present are at least one cation selected from the group consisting of Li⁺, Na⁺, K⁺, Mg²⁺, Ca²⁺, Al³⁺, NH₄⁺, primary ammonium ions, secondary ammonium ions, tertiary ammonium ions and quaternary ammonium ions.

5. The composition according to claim 1, comprising:
50% by weight to 99% by weight of glucolipid anions, wherein % by weight refers to all anions except OH⁻ present in the composition.

6. The composition according to claim 1, comprising:
1% by weight to 30% by weight of GL-C8C10, wherein GL-C8C10 is a glucolipid of formula (I) wherein one of the radicals R¹ and R² is (CH₂)₄—CH₃ and the remaining radical R¹ or R² is (CH₂)₆—CH₃,
wherein the percentages by weight refer to a sum of all of the glucolipids of formula (I) present.

7. The composition according to claim 1, comprising:
0.5% by weight to 20% by weight of GL-C10C12:1, wherein GL-C10C12:1 is a glucolipid of formula (I) wherein one of the radicals R¹ and R² is (CH₂)₆—CH₃ and the remaining radical R¹ or R² is (CH₂)₈—CH₃, and wherein the radical that is (CH₂)₈—CH₃ is an unbranched, unsubstituted hydrocarbon radical with 1 double bond,
wherein the percentages by weight refer to a sum of all of the glucolipids of formula (I) present.

8. The composition according to claim 1, comprising:
0.5% by weight to 20% by weight of GL-C10C12, wherein GL-C10C12 is a glucolipid of formula (I) wherein one of the radicals R¹ and R² is (CH₂)₆—CH₃ and the remaining radical R¹ or R² is (CH₂)₈—CH₃,
wherein the percentages by weight refer to a sum of all of the glucolipids of formula (I) present.

9. The composition according to claim 1, comprising:
1% by weight to 30% by weight of GL-C8C10, wherein GL-C8C10 is a glucolipid of formula (wherein one of the radicals R¹ and R² is (CH₂)₄—CH₃ and the remaining radical R¹ or R² is (CH₂)₆—CH₃;
0.5% by weight to 20% by weight of GL-C10C12:1, wherein GL-C10C12:1 is a glucolipid of formula (I) wherein one of the radicals R¹ and R² is (CH₂)₆—CH₃ and the remaining radical R¹ or R² is (CH₂)₈—CH₃, and wherein the radical that is (CH₂)₈—CH₃ is an unbranched, unsubstituted hydrocarbon radical with 1 double bond; and
0.5% by weight to 20% by weight of GL-C10C12, wherein GL-C10C12 is a glucolipid of formula (I) wherein one of the radicals R¹ and R² is (CH₂)₆—CH₃ and the remaining radical R¹ or R² is (CH₂)₈—CH₃;
wherein the percentages by weight refer to a sum of all of the glucolipids of formula (I) present.

10. The composition according to claim 1, comprising:
0% by weight to 5% by weight of GL-C10, wherein GL-C10 is a glucolipid of formula (I) wherein one of the radicals R¹ and R² is (CH₂)₆—CH₃, and
wherein the percentages by weight refer to a sum of all of the glucolipids of formula (I) present.

11. A method for preparing a formulation containing glucolipids, comprising:
a) providing a composition comprising glucolipids according to claim 1,
b) providing at least one further formulation component,
c) mixing the composition comprising glucolipids and the at least one further formulation component and adjusting a glucolipid content of the total composition to 0.5% by weight to 20% by weight of glucolipids by adding water, wherein the weight percentages refer to the total formulation.

12. The method according to claim 11, wherein the at least one further formulation component provided in b) is at least one selected from the group consisting of emollients, emulsifiers, thickeners/viscosity regulators/stabilizers, UV photoprotective filters, antioxidants, hydrotropes (or polyols), solids and fillers, film formers, pearlescent additives, deodorant and antiperspirant active ingredients, insect repellents, self-tanning agents, preservatives, conditioners, perfumes, dyes, odour absorbers, cosmetic active ingredients, care additives, superfatting agents, surfactants, and solvents.

13. A salt of at least one glucolipid, wherein said salt comprises at least one cation selected from the group consisting of Na⁺, Li⁺, K⁺, Mg²⁺, Ca²⁺, Al³⁺, NH₄⁺, primary ammonium ions, secondary ammonium ions, tertiary ammonium ions and quaternary ammonium ions.

14. The composition according to claim 1, comprising:
40% by weight to 50% by weight of at least one glucolipid of formula (I) or salts thereof; and
50% by weight to 60% by weight of water.

15. The composition according to claim 1, wherein the pH of the composition at 25° C. is from 4.5 to 5.4.

16. The composition according to claim 3, comprising:
at least 90% by weight of glucolipids of formula (I).

17. The composition according to claim 5, comprising:
85% by weight to 90% by weight, of glucolipid anions.

18. The method according to claim 11, wherein in c), the glucolipid content is adjusted to from 3.0% by weight to 12% by weight.
